(19) United States
(12) Patent Application Publication
(10) Pub. No.: US 2013/0245598 A1
(43) Pub. Date: Sep. 19, 2013

(54) PHOTOACTIVE VITAMIN NANOPARTICLES FOR THE TREATMENT OF CHRONIC WOUNDS

(71) Applicant: Patty Fu-Giles, University Park, IL (US)

(72) Inventor: Patty Fu-Giles, University Park, IL (US)

(21) Appl. No.: 13/810,555

(22) PCT Filed: Jul. 21, 2011
(86) PCT No.: PCT/US11/44819

§ 371 Date: Mar. 12, 2013

Related U.S. Application Data
(60) Provisional application No. 61/366,350, filed on Jul. 21, 2010.

(51) Int. Cl. A61K 41/00; A61N 5/06

(52) U.S. Cl. CPC A61K 41/0057; A61K 41/0071; A61N 5/062
USPC 604/501; 514/251; 514/52; 514/184

(57) ABSTRACT

The preparation and use of photosensitizer-containing nanoparticles including photoactive vitamin-containing nanoparticles as photodynamic antimicrobial agents are disclosed for tissue repair including the treatment of chronic wounds.

### CROSS-REFERENCE TO RELATED APPLICATION

This application claims priority from U.S. Provisional Patent Application Ser. No. 61/366,350, filed Jul. 21, 2010, the disclosure of which is incorporated herein by reference.

### TECHNICAL FIELD

The present invention relates to photosensitizer-containing nanoparticles and, in particular, to photoactive vitamin-containing nanoparticles as photodynamic antimicrobial agents for tissue repair including the treatment of chronic wounds.

### BACKGROUND OF THE INVENTION

Chronic wounds are a worldwide health problem, in part, due to a lack of adequate methods of treatment. Moreover, incorrect diagnosis, overuse of systemic antibiotics and ineffective use of compression therapy frequently aggravate the complications. In 2010, more than 7 million people worldwide suffered from chronic wounds, and the projected annual increase is at least 10 percent. Moreover, approximately 80,000 people undergo amputation each year due to wounds that do not heal properly. The expense in lost manpower, hospitalizations, debilitation and even death is costly on a global basis.

Conventional methods of killing bacteria (including antibiotics and disinfection) are often ineffective due to increasing rates of multidrug resistance and can cause additional damage to surrounding tissue, thus inhibiting recovery and overall successful surgical outcomes. The huge doses of antimicrobials required to treat patients who are already immunosuppressed are medically undesirable, and are increasingly ineffective.

Research has indicated that pathogenic biofilms are the primary hindrance to wound healing. Bacterial species within biofilms are exceptionally resistant to many traditional therapies. Photodynamic antimicrobial chemotherapy (PACT) is a potential alternative antibacterial, antifungal and antiviral treatment for drug-resistant microorganisms for the treatment of chronic wounds. It is very unlikely that organisms will develop resistance to cytotoxic reactive molecules such as reactive oxygen species (ROS) generated by the photosensitizers. However, a major limitation of this technique is the uptake kinetics of photosensitizers by the microorganisms.

It has been demonstrated that a combination of several bacteria species frequently colonize chronic wounds to form highly persistent biofilm communities. Chronic wound pathogenic biofilms are host-pathogen environments in which many bacteria species co-exist and cohabitate to promote their own survival. There is a common misconception that systemic antibiotics alone can treat these infections. Use of antibiotics alone is often harmful, because bacteria develop that are resistant to the antibiotic being used.

Photodynamic therapy (PDT) is a treatment regimen related to PACT that uses the combination of light and nontoxic drugs to destroy specific target cells. After the inactive and nontoxic drug is applied topically or is injected, the drug localizes in the selected tissue and can only be activated by irradiation with certain wavelengths of light. When these photosensitive drugs are activated, they can produce highly reactive intermediates and ultimately lead to the selective death of targeted cells without affecting normal tissue. Currently, PDT is being used primarily in the treatment of cancer. However, several studies have shown that PDT can also be effectively utilized in antimicrobial management. (Wainwright, M. (1998); Chen et al. (2002); Douglas, L. J. and Wainwright et al. (2004)). Research interest in antimicrobial PDT diminished for many years due to the widespread availability of antibiotics. However, in recent years, the emergence of antibiotic-resistant bacterial strains including methicillin-resistant Staphylococcus aureus (MRSA) and vancomycin-resistant Enterococcus faecalis has renewed interest in alternative treatments.

It has been established that there are specific major populations of bacteria that are included in all chronic wound biofilms. Species including Staphylococcus, Pseudomonas, Peptoniphilus, Enterobacter, Stenotrophomonas, Finegoldia and Serratia spp are found to be common chronic wound pathogens. (Daróczy et al.). Bacterial biofilms are highly organized microbial communities living within a protective extracellular matrix. They are difficult to detect and highly resistant to immune or antibiotic elimination. It has been suggested that biofilm presence may contribute to the intractable inflammatory processes seen in chronic wounds. Several clinical studies have shown a statistically significant (p<0.001) correlation between chronic wound patients developing biofilms (>60%) versus acute wound patients (6%) developing biofilms. Molecular analyses of chronic wound specimens have revealed the diverse polymicrobial communities and the presence of bacterial colonization. (Dowd et al.; Gjodsbol et al. and Ngo et al.).

Various metallic compounds have been investigated for their utility as PDT agents, most notably cisplatin in the treatment of cancer. (Lutterman et al.). The primary problems with cisplatin are its selective utility against various forms of cancer, its toxicity to human cells and the resistance that tumor cells may develop against it. Other metallic compounds have also been investigated for their photoreactive properties and their utility as PDT agents and, more specifically, as PACT agents. While many metallic compounds suffer from the same shortcomings as cisplatin, some do not exhibit the same side effects.

However, all such metallic compounds are difficult to synthesize based on their complex syntheses and are costly to manufacture. Naturally occurring compounds do not have the drawbacks associated with synthesis. Naturally occurring compounds have the added benefit of not harming normal tissue. That is, they remain inert until photoactivated—thus, they are exceptional candidates for use as PACT agents.

### SUMMARY OF THE INVENTION

The present invention relates to the preparation and use of compositions including photosensitizers and, in particular, photoactive vitamin-containing or phthalocyanine-containing nanoparticles as photodynamic antimicrobial chemotherapeutic (PACT) agents for tissue repair including the treatment of chronic wounds along with burns, soft tissue infections and infections of the skin and cornea. The compositions and methods described herein provide a significant improvement in the healing, for example, of chronic wounds using inexpensive, non-toxic photoactive vitamin nanoparticles as new photodynamic antimicrobial chemotherapeutic agents. In particular, the present invention addresses many of the major problems associated with post-wound infections by providing a new, fully characterized treatment that is based on well-known, relatively inexpensive and innocuous aqueous vitamin nanoparticles in combination with light therapy.

As described herein, non-toxic vitamins including riboflavin (vitamin B₂), cobalamin (vitamin B₁₂), phylloquinone (vitamin K₁) and menaquinone (vitamin K₂) are excellent photosensitizers that can produce singlet oxygen and free radicals upon irradiation. Other suitable photosensitizers include phthalocyanines, as described herein. A unique nanoemulsion is also disclosed for increasing the solubility of these otherwise hard to dissolve, hydrophobic vitamins and other photosensitizers to enable faster, more effective delivery to the target cells. The present invention provides a novel photodynamic chemotherapeutic regime for the treatment of chronic wound ulcers caused by microbial biofilms.

New strategies are needed for immediate use and PACT has been shown to be an effective alternative. In addition, other applications of this approach provide opportunities for other environmentally friendly uses such as bioremediating hazardous waste sites, biofiltering industrial water and forming biobarriers to protect soil and groundwater from contamination.

### BRIEF DESCRIPTION OF THE DRAWINGS

FIG. 1 shows an ethidium bromide stained agarose gel demonstrating the photocleavage of plasmid DNA from Form I (supercoiled) to Form II (nicked by riboflavin) after 30 min. incubation and 30 min. irradiation;

FIG. 2 shows the production of singlet oxygen by riboflavin as a function of irradiation time;

FIG. 3 is a transmission electron microscopy (TEM) image of vitamin K encapsulated nanoparticles;

FIG. 4 demonstrates the increased solubility of riboflavin in aqueous solution using a nanoemulsion formed according to the present invention;

FIG. 5 is a UV-Vis absorption spectrum comparing the absorption intensity as a function of wavelength of vitamin B₁₂ in aqueous solution (Line a) to the increased absorption intensity of vitamin B₁₂ entrapped within Surfynol-phosphocholine double-coated nanoparticles (Line b);

FIG. 6 is a fluorescence spectrum showing fluorescence intensity as a function of wavelength of various preparations of riboflavin including encapsulated nanoparticles; and

FIG. 7 shows copper phthalocyanine entrapped within Surfynol-poloxamer (Line 1) and Surfynol-phosphocholine (Line 2) double-coated nanoparticles.

### DETAILED DESCRIPTION OF THE INVENTION

As described herein, the present invention relates to a composition for the treatment of chronic wounds associated with the formation of biofilms comprising nanoparticles formed of a copolymer, the nanoparticles containing a therapeutically effective amount of at least one photosensitizer whereby irradiation of the photosensitizer provides for tissue repair. In one embodiment, the copolymer can comprise polyoxythylene and polyoxypropylene, and is preferably a poloxamer derivative.

In particular, copolymers useful in the practice of the present invention preferably include poloxamers including Poloxamer 407, Poloxamer 338, Poloxamer 237, Poloxamer 188 and Poloxamer 124; polysorbates including Polysorbate 20, Polysorbate 40, Polysorbate 60 and Polysorbate 80; polyethylene glycols including PEG 200, PEG 300 and PEG 400; polyoxyethylene ethers; poly (lactic-co-glycolic acids); and derivatives, mixtures and blends thereof.

Also, in a preferred embodiment, the photosensitizer is selected from the group consisting of riboflavin (vitamin B₂), cobalamin (vitamin B₁₂), phylloquinone (vitamin K₁) and menaquinone (vitamin K₂). The photosensitizer can also comprise a phthalocyanine selected from the group consisting of copper phthalocyanine, aluminum disulfonated phthalocyanine and zinc phthalocyanine.

The composition is preferably provided in a form suitable for topical administration which can include a liquid, lotion, cream or ointment. In the alternative, the composition can be applied as an aerosol using techniques well known in the art.

In a further embodiment, the present invention relates to a composition for tissue repair including the treatment of chronic wounds comprising first nanoparticles formed of a first copolymer, the first nanoparticles containing a therapeutically effective amount of a first photoactive vitamin, and second nanoparticles formed of a second copolymer, the second nanoparticles containing a therapeutically effective amount of a second photoactive vitamin, whereby irradiation of the first and second photoactive vitamins provides for tissue repair.

In an additional embodiment, a method is provided for the treatment of chronic wounds—and other skin conditions comprising administering a therapeutically effective amount of a pharmaceutical composition comprising nanoparticles formed of a copolymer or a polymer matrix and containing a therapeutically effective amount of at least one photosensitizer or photoactive vitamin to a wound of a subject and irradiating the subject to activate the photosensitizer or photoactive vitamin and provide for tissue repair.

As used herein, the term “nanoparticles” includes liposomes, micelles, inverse micelles, double-coated liposomes, nanoemulsions and a polymer matrix.

The polymer matrix can be used in any of the foregoing embodiments and can include one or more of the following polymers: a polyethylene glycol ether, a polyethylene glycol, a phosphocholine, a polyvinyl alcohol and a poloxamer. In a particularly preferred embodiment, the polymer matrix comprises a first polymer and a second polymer, wherein the first polymer is a polyethylene glycol ether and the second polymer is selected from the group consisting of a polyethylene glycol, a phosphocholine, a polyvinyl alcohol and a poloxamer including derivatives, mixtures and blends thereof.

A review of methods for producing nanoparticles including liposomes, micelles, inverse micelles and nanoemulsions is provided in Liposomes, Marc Ostro, ed., Marcel Dekker, Inc. New York, 1983, the relevant portions of which are incorporated herein by reference. See also Szoka, Jr. et al., (Ann. Rev. Biophys. Bioeng., 1980, 9:467), the relevant portions of which are also incorporated herein by reference.

As used herein, “therapeutically effective amount” refers to the amount of a photosensitizer or photoactive vitamin that produces the desired effect, in particular, tissue repair and healing in a patient or subject. The amounts will depend on a number of factors including the nature of the photosensitizer or photoactive vitamin, the physical characteristics of the subject and the extent of bacterial infection. A health professional of ordinary skill in the art can readily determine the range of an effective amount necessary to obtain the therapeutically desired result.

In practice, the subject is irradiated with light at a wavelength such that the photosensitizer or photoactive vitamin produces a cytotoxic effect relative to undesired organisms including the bacterial species identified herein to promote tissue repair and healing. Many photosensitizers produce free radicals and singlet oxygen which are highly reactive and can be toxic to organisms including bacterial species. The wavelength at which the photosensitizer is activated is determined using literature sources and direct measurement. Suitable light sources include any device capable of producing light of the wavelength required to activate the photosensitizer or photoactive vitamin.

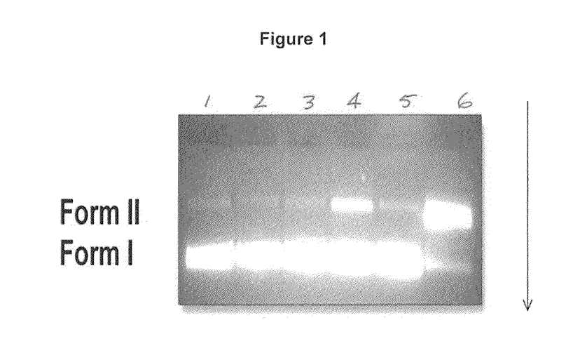

The present data demonstrates that riboflavin (7,8-dimethyl-10-ribityl-isoalloxazine or vitamin B₂) has an excellent DNA binding constant (K_{b}>10⁴ M⁻¹). As shown in FIG. 1, riboflavin can cleave DNA under irradiation with visible light (λ>395 nm). Ethidium bromide stained agarose gel shows the photocleavage of 120 μM plasmid DNA from Form I—supercoiled to Form II—nicked by riboflavin after 30 min incubation and 30 min of irradiation. From the left: Lane 1: DNA only, dark. Lane 2: DNA only, irradiated. Lane 3: DNA+25 μM riboflavin, dark. Lane 4: DNA+25 μM riboflavin, irradiated. Lane 5: DNA+50 μM riboflavin, dark. Lane 6: DNA+50 μM riboflavin, irradiated (18.8 mg/Kg).

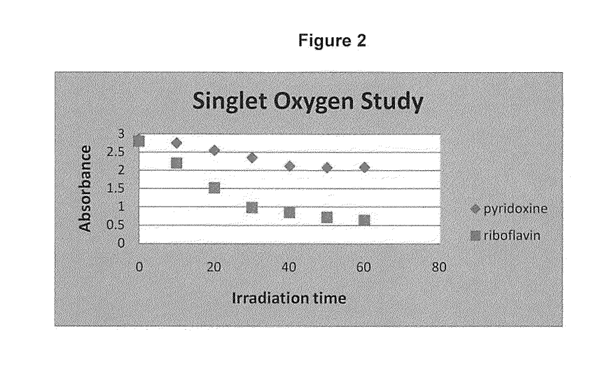

Referring to FIG. 2, a singlet oxygen study using anthracene-9,10-dipropionic acid (ADPA) as the singlet oxygen sensor shows that riboflavin is capable of producing a large quantity of singlet oxygen after only 15 minutes of irradiation.

Because most photoactive vitamins in nature are either hydrophobic or low in solubility, a biocompatible nano-emulsion has been developed in order to deliver lipophilic vitamins and other photosensitizers to target cells more effectively. A HLB (hydrophilic-lipophilic balance) emulsion system is used for preparing the oil-in-water nanoparticles. Vitamin F (a mixture of linoleic acid and alpha-linoleic acid) is used as a primary fatty acid and has a HLB of 14-15. It also meets the criteria of solubilizing oil in water. The HLB value needs to be between 13-18. Fatty acids suitable for use in the present invention include palmitic acid, linoleic acid, alpha-linoleic acid, oleic acid, transoleic acid, stearic acid, arachidic acid and tetracosanoic acid. Tween 20 (polysorbate 20 or polyoxyethylene (20) sorbitan monolaurate) has a HLB of 16.7 and is a suitable emulsifier.

Making Emulsion:

| | |
|---|---|
| Oil | 16% |
| Emulsifier | 4% |
| Water | 80% |

Primary Emulsion:

| | |
|---|---|
| Vitamin F with hydrophobic vitamin | 4 Parts |
| Water | 2 Parts |
| Tween 20 (Emulsifier) | 1 Part |

First, the hydrophobic vitamin is dissolved in the fatty acid (vitamin F). One part of Tween 20 is dissolved into 2 parts (w/v) of water. The prior two are combined to make the primary emulsion by adding vitamin F with the hydrophobic vitamin into aqueous Tween 20 dropwise with stirring to provide a clear emulsion. The primary emulsion is sonicated for about 1 hour. The primary emulsion is diluted 1:10 (by volume) to avoid aggregation and is then sonicated for about an additional 30 minutes.

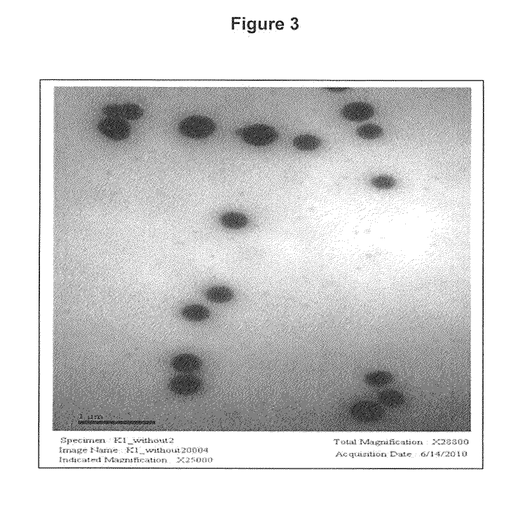

FIG. 3 is a transmission electron microscopy (TEM) image of vitamin K encapsulated nanoparticles according to the present invention.

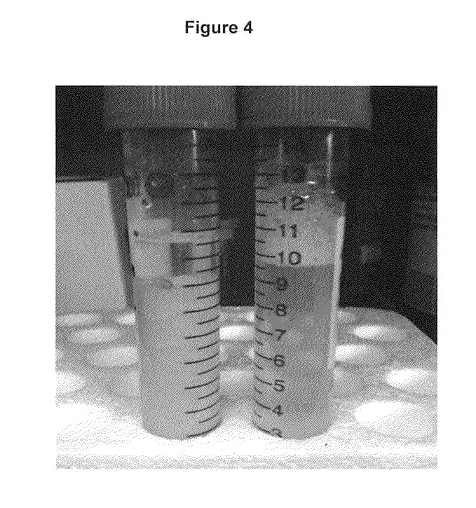

FIG. 4 shows that riboflavin solubility is increased more than 3-fold as a nanoemulsion. The vial on the left shows riboflavin precipitation in water at 250 μm riboflavin. The vial on the right shows a clear solution at 750 μM riboflavin as a nanoemulsion formed according to the present invention.

Antibacterial study has shown that after one hour incubation and 30 minutes irradiation with low intensity light (about 5 J/cm²), a concentration of riboflavin as low as 85 mg/Kg can cause 50% bacteria fatality. This is compared to the acute oral toxicity recorded on the MSDS of LD₅₀>10 g/Kg. Thus, the concentration used herein for eradicating bacteria would have no observable effect on normal human cells. The same result was also obtained in the phylloquinone study. Table 1 shows the complete summary of the bacterial study with LD₅₀ values in mg/Kg.

TABLE 1
LD₅₀ (lethal dose 50 in mg/Kg) of riboflavin and phylloquinone
in both irradiated and dark conditions. Acute oral systematic
toxicity of both was cited from the MSDS.

| | Human Skin | | E. coli | | Acute Oral |
|---|---|---|---|---|---|
| | Dark | Irradiated | Dark | Irradiated | Toxicity |
| Riboflavin | No Effect | 2,100 | 197 | 85 | >10,000 |
| Phyllo- | No Effect | 18,779 | 1,100 | 423 | 25,000 |
| quinone | | | | | |

### Materials and Methods

### Nanoemulsion and Nanoparticles

In general, neutral, anionic and cationic photosensitizers can efficiently eliminate Gram-positive bacteria. The porous cell wall of Gram-positive bacteria allows most photosensitizers to cross. However, only hydrophilic cationic photosensitizers can kill Gram-negative bacteria. The cell envelope (outer membrane) of Gram-negative bacteria forms an effective permeability barrier between the cell and its environment. This has led to intensive research on particulate delivery systems to overcome this situation. Studies have shown that using nanoemulsions or nanoparticles as carriers for biomedical applications can improve efficacy in solubilizing, protecting and targeting microorganisms for specified delivery. The present invention provides a significant advancement in tissue repair including chronic wounds due to biofilm infection.

In particular, the formulations of the following Examples entrap hydrophobic photosensitizers inside nanoparticles. Two photoactive, but hydrophobic, vitamins are described as representative photosensitizers. A significant reason for using a vitamin as a PACT agent is its non-toxic nature towards human tissues. Riboflavin (vitamin B₂) produces a significant amount of singlet oxygen when irradiated. However, it is not very soluble in water (water solubility=0.1 mg/ml). Phylloquinone (vitamin K₁) is a known fat soluble vitamin that does not dissolve in water. In order to make vitamins including riboflavin and phylloquinone suitable as PACT agents, it is necessary to increase their water solubility. According to the present invention, a hydrophobic vitamin is first encapsulated inside polymeric nanoparticles, and the nanoparticles are then suspended in water.

### Example 1

Riboflavin (3.0 mg) is dissolved in 10 ml of ethyl acetate to form an 800 μM (micromolar) organic phase. 500 μM palmitic acid (surfactant) is dissolved in 20 ml of water to form an aqueous phase. Palmitic acid is difficult to dissolve in water and must be sonicated for about 20 min. AOT (sodium 1,4-bis[(2-ethylhexyl)oxy]-1,4-dioxobutane-2-sulfonate) or PVA (polyvinyl alcohol) can be used instead, or palmitic acid can be dissolved in ethyl acetate. PVA is also difficult to dissolve in water at room temperature. The aqueous solution needs to be heated to about 80° C. for the PVA to dissolve. Reduce the temperature of the solution to about 50° C. before performing the next step.

The organic phase is added to the water phase dropwise with constant stirring over low heat. Ethyl acetate is very volatile and has a low boiling point of 77° C. It can be removed from the sample by gentle heating in a hot water bath. Stirring is continued until all of the ethyl acetate evaporates (some water also evaporates). The final volume should be less than 20 ml (the original water volume). 2% w/v high molecule weight PEG (polyethylene glycol) and 0.8% w/w Carbopol Ultrez 10NF polymer (The Lubrizol Corporation, Wickliffe, Ohio) are added as stabilizers and thickening agents.

### Example 2

PLGA-PEG block copolymer is used in this formulation. (Intl. J. Pharmaceutics, 1996, 138:1-12.) Riboflavin (3.0 mg) and 100 mg of poly(lactic-co-glycolic acid) (PLGA) are dissolved in 10 ml of ethyl acetate with constant stirring to form an organic phase. 58 mg of polyethylene glycol (PEG 200) is dissolved in 20 ml of water to form an aqueous phase. The above organic phase is added to the above aqueous phase dropwise with constant stirring. Ethyl acetate is very volatile and has a low boiling point of 77° C. It is removed from a sample by gentle heating in a hot water bath. The final product is an amphiphilic PLGA-PEG copolymer which forms micelles having a PLGA hydrophobic core and a PEG shell in water. 0.8% w/w Carbopol Ultrez 10NF polymer (Lubrizol) is added as a stabilizer and thickening agent.

### Example 3

This formulation entraps a hydrophilic photosensitizer inside the nanoparticle. A water-oil-water (w/o/w) double emulsion method is developed to entrap hydrophilic cobalamin (vitamin B₁₂) inside double-coated nanoparticles. Cobalamin (vitamin B₁₂) absorbs visible light and thus is an ideal candidate as a PACT agent. It also has a water solubility of 12.5 mg/ml.

Vitamin B₁₂ (6.0 mg) is dissolved in 5 ml of water to form a first (3 mM) aqueous phase. 4 ml of Surfynol 465 is dissolved in 10 ml of ethyl acetate to form an organic phase. The aqueous phase is added dropwise into the organic phase with constant stirring. Reverse micelles are formed in this step as a water-in-oil emulsion. Surfynol 465 is a surfactant comprising a polyethylene glycol ether (or acetylenic diol) supplied by Air Products Ltd. Other suitable polymers for use in the present invention include Surfynol 420, Surfynol 440, Surfynol 480 and Surfynol 485.

The final water phase is prepared by dissolving high molecular weight PEG (MW>2000 g/mol), phosphocholine, PVA or Poloxamer 407 in 10 ml of water. Poloxamer 407 is a hydrophilic non-ionic surfactant which comprises a triblock copolymer consisting of a central hydrophobic block of propylene glycol flanked by a pair of hydrophilic blocks of polyethylene glycol. Poloxamer 407 is also known by the BASF trade name Pluronic F 127.

The above water-in-oil emulsion is added dropwise to the final aqueous phase with constant stirring. Stirring is continued until all of the ethyl acetate evaporates. A double emulsion is formed in this step. Surfynol-PEG, Surfynol-phosphocholine, Surfynol-PVA and Surfynol 465-Poloxamer 407 are suitable pairs for hydrophobic-hydrophilic double emulsion. 0.8% w/w Carbopol Ultrez 10NF polymer (Lubrizol) is added as a stabilizer and thickening agent.

The first generation porphyrins investigated in the past for photodynamic therapy were based on chemically-modified natural hematoporphyrins. Such compounds have certain limitations including weak absorption in the phototherapeutic window and poor specificity towards malignant and healthy tissues. The second generation of photosensitizers was primarily based on engineered, synthetic and semi-synthetic porphyrins with various expanded substituents on the pyrrole rings and at the methylene bridges. The optical properties for therapy have improved in the second generation photosensitizers, but delivery to the target tissue is still a relatively passive process.

As described herein, several approaches improve the direct targeting and increase reactive species generation of PACT agents. The photosensitizers together with a direct delivery mechanism are known as the third generation of photosensitizers or smart drugs. Also, because most porphyrin and phthalocyanine derivatives are not water soluble, oil-in-water nanoemulsion and nanoparticle formulations have been developed, as described herein, to promote drug delivery.

### Example 4

Thirty (30) mg copper phthalocyanine (CuPc) and 4.0 ml of Surfynol 465 are dissolved in 10 ml of ethyl acetate over low heat with constant stirring to form an organic phase. 270 mg high molecular weight PEG (MW>2000 g/mol) is dissolved in 50 ml of water to form an aqueous phase. The organic phase is added dropwise to the aqueous phase with vigorous stirring until all of the ethyl acetate evaporates. Sonicate for about 15 minutes.

### Example 5

Six (6) mg of copper phthalocyanine (CuPc) and 2.0 ml of Surfynol 465 are dissolved in 10 ml of ethyl acetate over low heat with constant stirring to form an organic phase. 0.2 g of Poloxamer 407 is dissolved in 20 ml of water to form an aqueous phase. The organic phase is added to the aqueous phase with vigorous stirring over low heat until all of the ethyl acetate has evaporated. The solution is then degassed to remove the foam, and is sonicated for about 15 minutes.

### Example 6

Six (6) mg (5.5 μmole) of copper phthalocyanine (CuPc) and 2.0 ml of Surfynol 465 non-Ionic surfactant with low foaming characteristics are dissolved in 20 ml of ethyl acetate over low Heat with constant stirring to form an organic phase. 11 μmole of o-ctadecylphosphoryl)choline is dissolved in 20 ml of water to form an aqueous phase. The organic phase is added to the aqueous phase dropwise with vigorous stirring. Additional water is added with stirring and the total volume is brought to 100 ml. The solution is stirred overnight until all of the ethyl acetate has evaporated and the volume is reduced to 20 ml. The solution is then degassed to remove a foam and is sonicated for 15 minutes.

### Example 7

Riboflavin (3 mg) and 0.168 g of PLGA are dissolved in 10 ml of ethyl acetate under low heat with constant stirring to form an organic phase. 1 ml of Triton X-100 (HLB 11.6) is added to 10 ml of PBS to form an aqueous phase. Triton X-100 is a non-ionic surfactant having a hydrophilic polyethylene oxide group and a hydrocarbon lipophilic or hydrophobic group. It is manufactured by the Dow Chemical Company (Midland, Mich.) and is related to the Pluronic range of detergents marketed by BASF Corporation. The organic phase is added to the aqueous phase with vigorous stirring over low heat until all of the ethyl acetate has evaporated, and the solution is sonicated for about 15 minutes.

### Stabilizer and Thickening Agent

PEG and Poloxamer are well known stabilizers. Thus, no additional stabilizer is needed in some of the present formulations. Carbomer, a synthetic high molecular weight polymer of acrylic acid, can be used as an additional stabilizer and also as a thickening agent to increase the viscosity of the formulations.

### Cell and Bacteria Study

Each formulation is tested for its ability to deliver the PACT drugs into bacteria. The cell penetration studies are carried out using human dermal fibroblast cells. After about 2 hours incubation with the nanoparticles or nanoemulsion, the cell membrane is lysed using 5% N-lauryl sacrosine sodium salt solution. Fluorescent or UV-Vis readings are compared before and after cell lysis.

### Cytotoxicity and Photocytotoxicity

Toxicity of the formulations towards human skin cells is examined by previously published methods. (Fu-Giles et al., Photochemistry and Photobiology, 2005, 81:89-95.)

### Results

### Nanoparticles

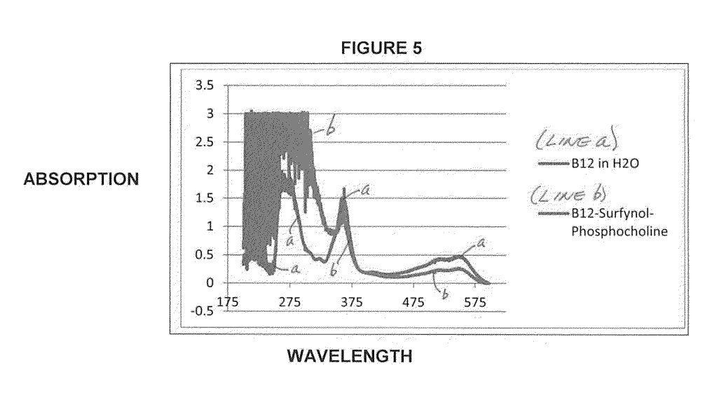

Referring to FIG. 5, line a represents the typical UV-Vis spectrum of aqueous vitamin B₁₂. It shows a π to π* transition peak at 272 nm, a n to π* transition peak at 370 nm and a MLCT (Metal-to-Ligand Charge-Transfer) transition peak at 570 nm. A MLCT transition is an electronic transmission of a metal complex that corresponds to excitation populating an electronic state in which considerable electron transfer from the metal to the ligand has occurred. Line b shows decreasing absorption intensity compared to line a at the same concentration (50 μM) of vitamin B₁₂. The Figure clearly indicates that vitamin B₁₂ is entrapped within the double-coated nanoparticles. The significant UV absorption below 300 nm is due to the absorbivity of phophocholine which has a λₘₐₓ of 225 nm.

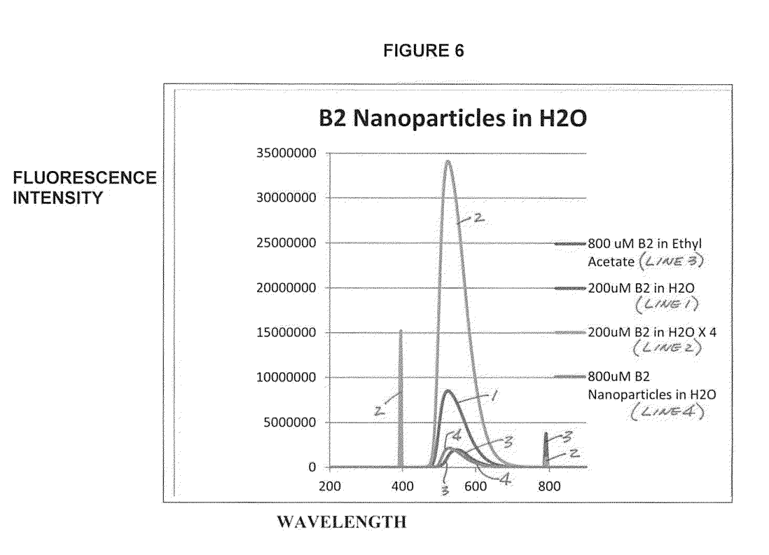

FIG. 6 shows that vitamin B₂ is encapsulated within palmitic acid nanoparticles (Example 2). It is well known that vitamin B₂ (riboflavin) is not very soluble in water; it reaches saturation at 200 μM. Line 1 shows the fluorescence of 200 μM riboflavin in aqueous solution. Line 2 demonstrates the intensity of 800 μM riboflavin in water. Line 3 shows 800 μM riboflavin in ethyl acetate. Line 4 shows 800 μM riboflavin encapsulated within the nanoparticles, then suspended in water. The hypochromic effect of Line 4 compared to Line 3 (both 800 μM) indicates increasing hydrophilicity. Upon comparing Line 2 and Line 4, it is evident that riboflavin is encapsulated within the nanoparticles.

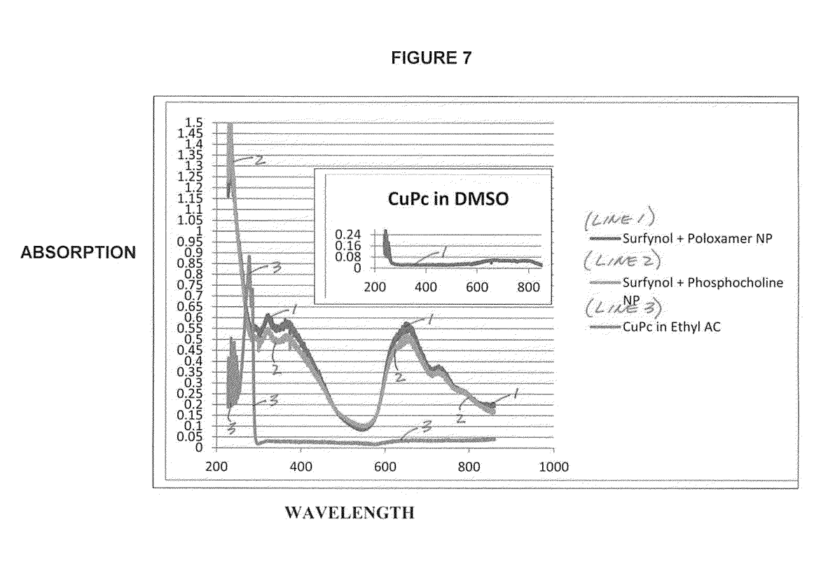

FIG. 7 shows copper phthalocyanine (CuPc) entrapped within Surfynol-Poloxamer (Line 1) and Surfynol-phosphocholine (Line 2) double-coated nanoparticles. CuPc is a hydrophobic molecule, when dissolved in a non-polar solvent such as ethyl acetate, the electronic absorption spectra shows only a strong π to π* peak (Line 3). Electronic transitions n to π* and MLCT start to take place only when the polarity of the solvent increases. Dimethyl sulfoxide (DMSO) has a polarity of 7.2. The inset graph shows that when CuPc is dissolved in DMSO, the absorption spectra starts to show a MLCT band due to the increased excited state dipole moment. The electronic absorption shows very different transitions in the visible region (Lines 1 and 2) when CuPc nanoparticles are suspended in water (polarity=10.2) versus when the nanoparticles are suspended in the non-polar solvent (Line 3).

### PUBLICATIONS

1. Wainwright, M., “Photodynamic antimicrobial chemotherapy (PACT).” J. Antimicrob. Chemther., 1998, 42, 13-28.
2. Biel, M. A., “Photodynamic therapy and the treatment of head and neck neoplasia.”Laryngoscope, 1998, 108, 1261-1265.
3. Frelon S., Douki T., Cadet J., “Radical oxidation of the adenine moiety of nucleoside and DNA:2-hydroxy-2′-deoxyadenosine is a minor decomposition product.” Free Radic. Res., 2002, 36, 499-508.
4. Chen, J., Keltner, L., Christophersen, L., Zheng, F., Krouse, M., Singhal, A., “New technique for deep light distribution in tissue for photodynamic therapy.” Cancer J., 2002, 8, 154-163.
5. Douglas, L. J., “Candida biofilm and their role in infection.” Trends Microbiol., 2003, 11, 30-36.
6. Brown, S. B., Brown, E. A., Walker, I., “The present and future role of photodynamic therapy in cancer treatment.” Lancet Oncol., 2004, 5, 497-508.
7. Wainwright M., Crossley, K. B., “Photosensitizing agents—circumventing resistance and breaking down biofilms: a review.” Int. Biodeterior. Biodegrad., 2004, 53, 119-126.
8. Chen, H. M., Yu, C. H., Yeh, C. Y., Tsai, T., Chiang, C. P., “Successful treatment of oral verrucous hyperplasia and oral leukoplakia with topical 5-aminolevulinic acid-mediated photodynamic therapy.” Laser Surg. Med., 2005, 37, 114-122.
9. Allison, R. R., Bagnato, V. S., Cuenca, R., Downie, G. H., Sibata, C. H., “The future of photodynamic therapy in oncology.” Future Oncol., 2006, 2, 53-71.
10. Komerik, N., MacRobert, A. J., “Photodynamic therapy as an alternative antimicrobial modality for oral infections.” J. Environ. Pathol. Toxicol. Oncol, 2006, 25, 487-504.
11. Konopka, K., Goslinski, T., “Photodynamic Therapy in Dentistry.” J. Dent. Res., 2007, 86, 694-707.
12. Daróczy, J., Quality Control in Chronic Wound Management: The Role of Local Povidone-Iodine (Betadine®) Therapy, Dermatology, 2006; 212 (Suppl. 1):82-8.
13. Dowd, S. E., Sun, Y., Secor, P. R., Rhoads, D. D., Wolcott, B. M., James, G. A. and Wolcott, R. D., Survey of bacterial diversity in chronic wounds using Pyrosequencing, DGGE, and full ribosome shotgun sequencing.” BMC Microbiology, 2008, 8.
14. Gjodsbol, K., Christensen, J. J., Karlsmark, T., Jorgensen, B., Klein, B. M., Krogfelt, K. A., “Multiple bacterial species reside in chronic wounds: a longitudinal study.” Int. Wound J., 2006, 3, 225-231.
15. Ngo, Q., Vickery, K., Deva, A. K., “P21 role of bacterial biofilms in chronic wounds.” ANZJ Surg., 2007, 77, 1:A66.
16. Lutterman, D. A., Fu, Patty K.-L., Turro, C., “cis-[Rh2(i-O2CCH3)2(CH3CN)6]2+ as a Photoactivated Cisplatin Analog”, J. Am. Chem. Soc., 2006, 128, 739.
17. Long, E. C. and Barton, J. K., Acc. Chem. Res., 1990, 23, 271.
18. Dougherty, G. and Prigram, W. J., Crit. Rev. Biochem., 1982, 12, 103.
19. Berman, H. M. and Young, P. R., Ann. Rev. Biophys. Biwng., 1981, 10, 87.
20. Cantor, C. and Schimmel, P. R., Biophysical Chemistry, Vol. 2, 1980, 398.
21. Hui-Juan, Y., Hui, C., Long, J., Lu-Ying, L., Shu-Mei, H., Liang-Nian, J., “Single oxygen-mediated DNA photocleavage of a di-bithiazolyl ruthenium(II) complex [Ru(btz)₂(dppz)]²⁺”, Inorganic Chemistry Communications, May 2008, Vol. 11, 5, 553-556.
22. Gupta, T., Patra, A. K., Dhar, S., Nethaji, M., Chakravarty, A. R., “Effect of copper-sulphur bond on the DNA photo-cleavage activity of 2-(methylthio)ethylpyridine-2-carbaldimine copper(II) complexes”, J. Chem. Sci., March 2005, Vol. 117, No. 2, 123-132.
23. Stoilova, 0., Jerome, C., Detrembleur, C., Mouithys-Mickalad, A., Manolova, N., Rashkov, I., Jerome, R., “New Nanostructured Materials Based on Fullerene and Biodegradable Polyesters.”, Chem. Mater., 2006; 18, 4917-4923.
24. Viola, G., Ihmels, H., Krauber, H., Vedaldi, D., and Dall' Acqua, F., “DNA-binding and DNA-photocleavaging properties of 12a, 14adiazoniapentaphene”, ARKIVOC, 2004, 219-230.
25. Holmlin, R. E. and Tong, R. T.; Barton, J. K., J. Am. Chem. Soc., 1998, 120, 9724.

All publications listed or referred to herein are incorporated by reference to the extent those publications are not inconsistent herewith.

Although the present invention has been disclosed with respect to particular nanoparticles, photosensitizers, vitamins, formulations and methods, it will be apparent that a variety of modifications and changes can be made without departing from the scope and spirit of the invention, as described and claimed herein.

What is claimed is:

1. A composition for the treatment of chronic wounds comprising nanoparticles formed of a copolymer, the nanoparticles containing a therapeutically effective amount of at least one photosensitizer whereby irradiation of the photosensitizer provides for tissue repair.

2. The composition according to claim 1 wherein the copolymer comprises polyoxyethylene and polyoxypropylene.

3. The composition according to claim 1 wherein the copolymer is selected from the group consisting of poloxamers, polysorbates, polyethylene glycols, polyethylene glycol ethers, phosphocholines, polyvinyl alcohols, poly(lactic-co-glycolic acids) and derivatives, mixtures and blends thereof.

4. The composition according to claim 1 wherein the photosensitizer is selected from the group consisting of riboflavin (vitamin B₂), cobalamin (vitamin B₁₂), phylloquinone (vitamin K₁), menaquinone (vitamin K₂), and a phthalocyanine.

5. The composition according to claim 4 wherein the phthalocyanine is selected from the group consisting of copper phthalocyanine, aluminum disulfonated phthalocyanine and zinc phthalocyanine.

6. The composition according to claim 1 wherein the composition is in a form suitable for topical administration.

7. The composition according to claim 1 wherein the composition is in a form selected from the group consisting of liquid, lotion, cream or ointment.

8. A composition for the treatment of chronic wounds comprising nanoparticles formed of a copolymer, the nanoparticles containing a therapeutically effective amount of at least one photoactive vitamin whereby irradiation of the photoactive vitamin provides for tissue repair.

9. The composition according to claim 8 wherein the copolymer comprises polyoxyethylene and polyoxypropylene.

10. The composition according to claim 8 wherein the copolymer is selected from the group consisting of poloxamers, polysorbates, polyethylene glycols, polyethylene glycol ethers, phosphocholines, polyvinyl alcohols, poly(lactic-co-glycolic acids) and derivatives, mixtures and blends thereof.

11. The composition according to claim 8 wherein the vitamin is selected from the group consisting of riboflavin (vitamin B₂), cobalamin (vitamin B₁₂), phylloquinone (vitamin K₁) and menaquinone (vitamin K₂).

12. The composition according to claim 8 wherein the composition is in a form suitable for topical administration.

13. The composition according to claim 8 wherein the composition is in a form selected from the group consisting of liquid, lotion, cream or ointment.

14. A composition for the treatment of chronic wounds comprising first nanoparticles formed of a first copolymer, the first nanoparticles containing a therapeutically effective amount of a first photoactive vitamin, and second nanoparticles formed of a second copolymer, the second nanoparticles containing a therapeutically effective amount of a second photoactive vitamin, whereby irradiation of the first and second photoactive vitamins provides for tissue repair.

15. The composition according to claim 14 wherein the first and second copolymers comprise polyoxyethylene and polyoxypropylene.

16. The composition according to claim 14 wherein the first and second copolymers are selected from the group consisting of poloxamers, polysorbates, polyethylene glycols, polyethylene glycol ethers, phosphocholines, polyvinyl alcohols, poly(lactic-co-glycolic acids) and derivatives, mixtures and blends thereof.

17. The composition according to claim 14 wherein the first and second photoactive vitamins are selected from the group consisting of riboflavin (vitamin B₂), cobalamin (vitamin B₁₂), phylloquinone (vitamin K₁) and menaquinone (vitamin K₂).

18. The composition according to claim 14 wherein the composition is in a form suitable for topical administration.

19. The composition according to claim 14 wherein the composition is in a form selected from the group consisting of liquid, lotion, cream or ointment.

20. A method for the treatment of chronic wounds comprising:
administering an effective amount of a pharmaceutical composition comprising nanoparticles formed of a polymer matrix and containing a therapeutically effective amount of at least one photoactive vitamin to a wound of a subject; and
irradiating the subject to activate the photoactive vitamin and provide for tissue repair.

21. The method according to claim 20 wherein the polymer matrix comprises at least one polymer selected from the group consisting of a polyethylene glycol ether, a polyethylene glycol, a phosphocholine, a poly (lactic-co-glycolic acid), a polyvinyl alcohol and a poloxamer including derivatives, mixtures and blends thereof.

22. The method according to claim 20 wherein the polymer matrix comprises a first polymer and a second polymer, wherein the first polymer is a polyethylene glycol ether and the second polymer is selected from the group consisting of a polyethylene glycol, a phosphocholine, a poly(lactic-co-glycolic acid), a polyvinyl alcohol and a poloxamer including derivatives, mixtures and blends thereof.

23. The method according to claim 20 wherein the photoactive vitamin is selected from the group consisting of riboflavin (vitamin B₂), cobalamin (vitamin B₁₂), phylloquinone (vitamin K₁) and menaquinone (vitamin K₂).

24. The composition according to claim 20 wherein the composition is in a form suitable for topical administration.

25. The composition according to claim 20 wherein the composition is in a form selected from the group consisting of liquid, lotion, cream or ointment.
